(19) United States
(12) Patent Application Publication
(10) Pub. No.: US 2016/0228007 A1
(43) Pub. Date: Aug. 11, 2016

(54) SOLAR ENERGY HARVESTING, STORAGE, AND PROPAGATION IN NANOSCALE AND MICRO-SCALE STRUCTURES

(71) Applicant: Mike Shultz, Ames, IA (US)

(72) Inventor: Mike Shultz, Ames, IA (US)

(21) Appl. No.: 14/670,014

(22) Filed: Mar. 26, 2015

Related U.S. Application Data
(63) Continuation of application No. PCT/US13/61969, filed on Sep. 26, 2013.
(60) Provisional application No. 61/706,209, filed on Sep. 27, 2012.

(51) Int. Cl. A61B 5/00; A61K 9/00; H01L 51/42; A61K 9/48; H01L 51/00; G01N 21/64; A61K 49/00

(52) U.S. Cl. CPC A61B 5/0071; G01N 21/6486; A61B 5/0082; A61K 9/0014; A61K 49/0067; A61K 9/4825; H01L 51/0093; H01L 51/42

(57) ABSTRACT

The present invention relates generally to nanowires, nanospheres and microspherical structures derived from keratin, such as bird feathers. Such structures may be utilized to form biodegradable, organic, non-toxic solar cells for the purpose of propagating energy in a circuit of an electronic device. Structures of the present invention can also be utilized in pharmaceutical delivery devices and biosensing purposes, in particular, detecting cancerous cells in vivo.

### CROSS-REFERENCE TO RELATED APPLICATIONS

This application is a Continuation application of PCT/US13/61969 filed Sep. 26, 2013, which claims priority under 35 U.S.C. §119 to provisional application Ser. No. 61/706,209, filed Sep. 27, 2012, all of which are hereby incorporated in their entirety.

### FIELD OF THE INVENTION

The present invention relates generally to nanostructured solar cells composed of biodegradable, organic, non-toxic substances. More particularly, but not exclusively, the invention relates to nanowires, nanospheres and microspherical structures derived from a keratin substrate.

### BACKGROUND OF THE INVENTION

With global demands for energy increasing and environmental degradation due to combustion of fossil fuels, the need to produce affordable, sustainable, and carbon-free, environmentally safe energy is direr than ever. Recent developments in photo-conversion, utilizing dye sensitized and/or space-quantized nano-scale structured solar cells have the highest energy conversion efficiencies and; therefore, are the most promising technologies to satisfy these requirements.

Solar cells that incorporate nano-sized structures have a number of advantages over their bulk counterparts. When semiconductor structure size (nanoscale) approaches or becomes smaller than the distance between structures (de Broglie wavelength), photoelectrochemistry differs from that of bulk semiconductors, primarily due to the absence of field effects in nano-structured materials. Field effects arise from electrochemical potential differences between two dissimilar media (e.g., electrolyte-semiconductor) that when in electrical contact create a charge flowing across the interface resulting in rapid depletion of majority carriers. In nanoscale materials (quantum dots, quantum wires, and quantum wells), dimensions are quantized at levels smaller than that of the charge-field and field effects become absent or negligible, which results in increased electron-hole binding energy, exciton formation, and ultimately, increased energy conversion efficiencies. The highest energy conversion efficiency for “classical” silicon photovoltaic cells is ˜27%, while recently commercialized nano-structured solar cells are ˜45%.

Standard solar cells, as well as current nano-structured solar cells pose serious ecotoxicity risks. As a result, development of nano-structured solar cells composed of biodegradable, non-toxic substances would be a tremendous benefit. While nano-structured inorganic solar cells typically have greater conversion efficiencies as compared to organic, there exists significant room for improvement in the areas of increased energy conversion efficiency, reduced cost of production, and notably, reduced ecotoxicity.

On the other hand, organic semiconductors for use in solar cells have received considerable attention, but unfortunately not for potential benefits in reducing ecotoxicity. The term “organic” is somewhat misleading, as even the most efficient semiconductors contain many inorganic substances—such as fullerenes—that pose serious ecotoxicity risks. The advantage to current “organic” solar cells is their greatly reduced cost of production over inorganic solar cells. However, there are many obstacles hindering commercialization of organic solar cells. The main obstacles are poor sensitivity to red light, low charge mobility, loss of potential energy in charge-separation process, and imperfections in design structures. Organic solar cell energy conversion efficiencies are significantly lower than inorganic cells (organic 12% maximum vs. <45% for inorganic). Short-circuit densities are much lower than inorganic cells, as are Fill Factors (best is approximately 0.6 as opposed to 0.75 inorganic and 0.81 estimated here). V_{oc} (open circuit voltage) for organic and inorganic are similar (0.6-1.0 V vs. 0.7 V for crystalized silicon, and 0.72 for feathers). The most significant barrier to commercialization of organic solar cells is durability due to: a) oxidation of any conjugated organic materials in the presence of light and oxygen; b) mechanical stability; and, 3) loss of conductivity in the active-layer electrode interface. Thus, what is needed is an organic solar cell that achieves increased energy conversion efficiency, reduced ecotoxicity, low cost of production, and enhanced durability.

### SUMMARY OF THE INVENTION

Therefore, it is a principal object, feature, and/or advantage of the present invention to provide an organic solar cell that overcomes the deficiencies in the art.

It is another object, feature, and/or advantage of the present invention to provide semi conducting, solar light energy propagating nanowires, nanotubes, and nano and micro scale spheres of varying diameters derived from a keratin substrate.

It is yet another object, feature, and/or advantage of the present invention to provide nanostructures using inexpensive, organic, biodegradable materials that are inexpensive to manufacture.

It is still another object, feature, and/or advantage of the present invention to provide a solar cell derived from bird feathers.

It is further object, feature, and/or advantage of the present invention to provide nanostructures derived from bird feathers that emit blue light under near ultra-violet radiation.

It is still a further object, feature, and/or advantage of the present invention to provide a process to create or modify the properties of existing nanowires, nanospheres, microspheres and networks of these nanostructures by oxidative degradation such as via ultraviolet (“UV” or “UVA”) illumination of bird feathers.

It is another object, feature, and/or advantage of the present invention to provide bio-sensing applications and pharmaceutical delivery devices comprised of nanostructures derived from a keratin substrate, such as bird feathers, that are organic, biodegradable, non-toxic and maintain fluorescent properties for extended periods of time.

It is another object, feature, and/or advantage of the present invention to provide a protein molecule switch that can be used to control the flow of electromagnetic energy through a network of nano-structures or a solar cell derived from a keratin substrate.

These and/or other objects, features, and/or advantages of the present invention will be apparent to those skilled in the art. The present invention is not to be limited to or by these objects, features, and advantages. No single embodiment need provide each and every object, feature, or advantage.

According to one aspect of the present invention, semi conducting, solar light energy propagating nanowires, nanotubes, and nano and micro scale spheres of varying diameters are provided. The nanowires, nanotubes, and nano and micro scale spheres are derived from a keratin substrate, such as bird feathers, that efficiently absorb and propagate solar light over a broad range of wavelengths with minimal light loss. These nanostructures use inexpensive, organic, biodegradable materials, and are inexpensive to manufacture. The nanowires and spheres under high concentration will assemble into microspheres.

According to another aspect of the present invention, a solar cell derived from a keratin substrate, such as bird feathers, is provided. The solar cell comprises a network of nanowires that may also be comprised of an assemblage of nanospheres. The solar cell consists of a substrate layer of keratin organized in a series of interconnected hollow chambers that connect to a central shaft similarly comprised of interconnected hollow chambers. Light passes through the transparent upper side (oriented toward light) comprised of substrate and is harvested by a mesh of nanowires that line the inside of the chambers. These smaller nanowires merge into larger ones and pass through openings in chambers where they converge to a central shaft where light is propagated. The band gap and resulting photocurrent of solar cells may be modified by adding an underlying layer of highly absorbent material such as melanin.

According to still another aspect of the present invention, nanowires, nanospheres, and microspherical structures are provided that emit blue light under near ultra-violet radiation. Such structures are organic, biodegradable, non-toxic and maintain their fluorescent properties for extended periods of time. They are formed from, and can be attached to, a substrate of keratin such as bird feather keratin. The combination of these attributes makes them ideal candidates for bio-sensing applications and pharmaceutical delivery devices. In particular, their affinity for keratin makes them a candidate for detection and treatment of cancer as cancerous tumors contain large amounts of keratin. These nano and microstructures may contain large amounts of tryptophan and produce blue fluorescence under near UV illumination.

According to a further aspect of the present invention, a process is provided to create or modify the properties of existing nanowires, nanospheres, microspheres and networks of these structures by oxidative degradation of a keratin substrate. For instance, feather keratin may be degraded by UV irradiance, which causes the byproducts of keratin degradation to increase in concentration inside hollow chambers of the feather. As the concentration of these byproducts increases, these byproducts self-assemble into nanospheres, nanowires, networks of nanowires, and microspheres. This process may be slowed by the inclusion of a melanin sub-layer to absorb scattered photons not directly absorbed by nano and micro scale light gathering structures. The network of nanowires described above may be utilized in electronic devices for purpose of propagating energy in a circuit. Moreover, a protein molecule switch can be used to control the flow of electromagnetic energy through a network of nano-structures or a solar cell derived from feathers. This switch is turned on or off, or tuned by changes in the surrounding electromagnetic field.

### BRIEF DESCRIPTION OF THE DRAWINGS

FIG. 1 is an image of the right side of a scapular feather of a female Tree Swallow excited with a 310 nm laser.

FIG. 2 is an image of the right side of a scapular feather of a female Tree Swallow after exposure to UVA light for one week.

FIG. 3 is an image of a female Tree Swallow feather after UVA irradiation for one week showing micro-spheres (quantum dots) and nano sale light energy conducting tubes (nanowires).

FIG. 4 is an image of a focal plane of the same feather as in FIG. 3.

FIG. 5 is an image at higher magnification of the feather depicted in FIGS. 3 and 4 illustrating the network of nanowires in the feather shaft.

FIG. 6 is an image of microspherical structure (“quantum dot”) in the barbule of the same feather as depicted in FIGS. 3, 4, and 5.

FIG. 7 is a confocal microscopy image with a DAPI filter of the same feather as depicted in previous figures, post UVA irradiation for one week. DAPI is a fluorescent stain that binds strongly to A-T rich regions in DNA. It is used extensively in fluorescence microscopy.

FIG. 8 is an image of a feather barbule shaft with DAPI filter and UV laser illumination.

FIG. 9 is a scanning electron microscopy image of a Tree Swallow feather barb.

FIG. 10 is a scanning electron microscopy image of a female Tree Swallow feather barb that has been freeze-fractured.

FIG. 11 is an image at higher magnification of the same region as illustrated in FIG. 10.

FIG. 12 is an image at higher magnification of the same region as illustrated in FIG. 11 depicting nanowires and nanowire mesh lining the inside of the feather shaft.

FIG. 13 is another view of nanowires and mesh lining the inside of the feather shaft and barbules.

FIG. 14 are graphs illustrating female Tree Swallow crown reflectance and emittance taken at 1-second intervals for 3 minutes.

FIG. 15 are graphs illustrating oscillating current as depicted by photoluminescence when attached to a living bird as compared to a euthanized bird.

FIG. 16 are graphs depicting examples of pre and post mortem feather photoluminescence indicative of current flow in a system.

FIG. 17 are graphs depicting the typical photoluminescence of tree swallow feathers when exposed to an incident light source for several minutes continuously.

FIG. 18 depicts how current flow in a live bird is influenced by an electromagnetic field.

FIG. 19 depicts raman microscopy of a female Tree Swallow feather.

FIG. 20 depicts raman microscopy of nano-wires in Tree Swallow feathers as compared to raman profiles of keratin from several other taxa.

FIG. 21 depicts the similarities and differences between nanowire raman scattering profiles and that of carbon nanotubes.

FIG. 22 depicts simple dipeptides as building blocks for quantum dots and nanotubes.

FIG. 23 compares images of Tree Swallow feathers before and after one week of UVA radiation.

### DETAILED DESCRIPTION OF THE PREFERRED EMBODIMENTS

The present invention overcomes the deficiencies in the art by providing an organic solar cell derived from a keratin substrate, comprised entirely of organic, naturally occurring, biodegradable materials that are extremely durable. For instance, feathers are largely composed of keratin, as are the hair, nails and epidermis of mammals, and the scales of reptiles. In the absence of oxidative degradations, such as via ultra-violet light, feathers can persist undegraded for hundreds of years, as evidenced by museum collections and archeological finds. However, evidence suggests that when feather-derived solar cells are connected to a drain, the absorption of UV light does not result in the breakdown of feather keratin. Instead, peptide derived feather nano-structures propagate light energy through charge transfer without the release of oxygen free radicals that cause photo-oxidation and breakdown of keratin protein structure.

Nano-structures existing in feathers represent a unique situation. The field of nano-science has expanded exponentially in recent years, as the potential advantages of nanostructured solar cells have been recognized, such as by improving energy efficiency, reducing the size of electronic devices, use in biosensing applications, and the creation of quantum computers. However, naturally occurring nanostructures that perform functions of nanostructured solar cells are currently unknown to science. Thus, nanostructures and systems formed in feathers represent new opportunities for application. Such feather-derived nanostructures are utilized by birds to efficiently collect, store, and transport light energy to living tissue and drive biological processes. For example, keratin can be used and artificially constructed in a manner similar to the configuration of an avian feather to create solar cells.

One of these applications is the development of an organic solar cell with high efficiency. The highest energy conversion rate (n) of a solar cell is 43.5%, yet preliminary analyses suggest that energy conversion efficiencies of feather derived solar cells can be as high as n=80%. This is extraordinary—as given the rate of progress with current designs—it would take 25-30 years to reach 80% efficiency. The components of the solar cell described above derived from feathers have numerous additional applications. The nano-structured components include quantum dots, quasi-ballistic dots, nano-wires, and nanotubes that can be used to build transistors, computer processors, and electrical circuits for use in quantum computers, bio-sensing, bioassays, and others. Such components harness and propagate light energy in a controlled manner, possibly via plasmons. In addition to the electrochemical and photochemical properties described above, controlled plasmon propagation is an area of intensive work, as they are considered important in biosensing, molecule identification technologies, electronics, and computing. These nanostructures have the ability to form networks, transmit energy (and possibly plasmons) with high efficiency, emit light, and store energy. Moreover, such structures appear to have superior performance as compared to other leading designs, are biodegradable, non-toxic, and maintain a low material cost and cost of assembly. For these reasons, the present invention is likely to enable significant advances in sustainable energy sources, electronics, and health care.

Another embodiment of the present invention may be used in pharmaceutical delivery devices and biosensing purposes, in particular, detecting cancerous cells in vivo. However, it should be appreciated that other medical procedures and tests, besides detecting cancerous cells, can also be performed using the present invention. Currently utilized nanoscale spheres incorporate heavy metals into their design in order to achieve light absorption and photoluminescence levels necessary for their detection in biosensing. It is not well known how these substances are subsequently excreted by the body and therefore are still in the experimental state. Conversely, the quantum dots derived from keratin, such as from bird feathers, are non-toxic, have comparable or higher energy conversion efficiencies than inorganic ones, and the emission and fluorescence lifetimes are potentially longer and possibly significantly longer. These factors make keratin derived quantum dots ideal for biosensing applications. Quantum dots can also be engineered as core-shell dots, where a substance of lower energy state is encapsulated by that of a higher energy state. Such quantum dots derived from keratin could be used in drug delivery devices and engineered for such purposes. Moreover, keratin derived quantum dots could be used as a direct cancer treatment by delivering UV radiation directly to the cancerous cells.

Quantum dots derived from bird feathers originate from keratin and, thus, bind strongly to it. Consequently, feather derived quantum dots may concentrate at sites of where keratin levels are higher (e.g., cancerous tumors) allowing tumors to be detected with existing devices currently used for detecting quantum dots in vivo. In addition, keratin derived quantum dots could be engineered to encapsulate pharmaceuticals used for cancer treatment. When the quantum dot comes into contact with the cancerous tumor, the conformation of the dot may change and provide localized release of the pharmaceutical. Given that keratin derived quantum dots have a high affinity for keratin; topical solutions could be administered to the skin and hair. The quantum dots would traverse down hairs to capillary beds in the skin and carried throughout the circulation system to concentrate at tumor locations. Keratin derived quantum dots would have the added advantage of being non-toxic, organic substances.

The nano-structures described above are all derived from keratin and, therefore, would potentially be extremely cost effective to produce. Thus, these structures can be derived from generally any source of keratin. Keratin is one of the most abundant proteins in nature and could be acquired from wool, feathers, or even human hair for use light energy harvesting nano-structures and solar cells.

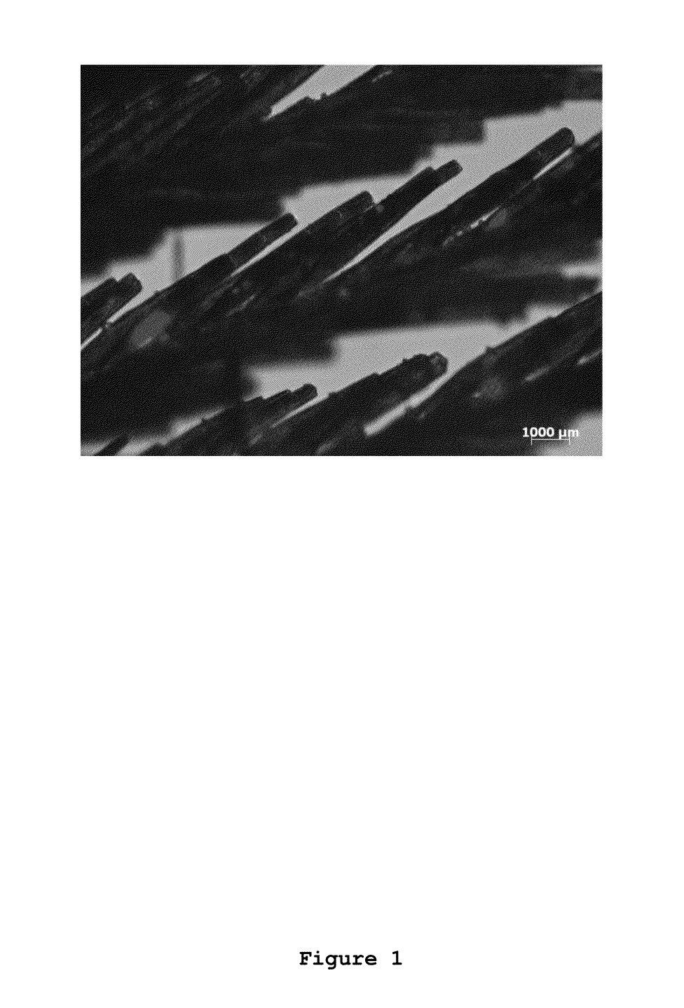

For instance, FIG. 1 is an image of a Female Tree Swallow right side scapular feather blue fluorescence excited with 310 nm laser, superimposed onto bright field image. As shown in FIG. 1, the blue fluorescence depicts locations of highly concentrated nanostructures.

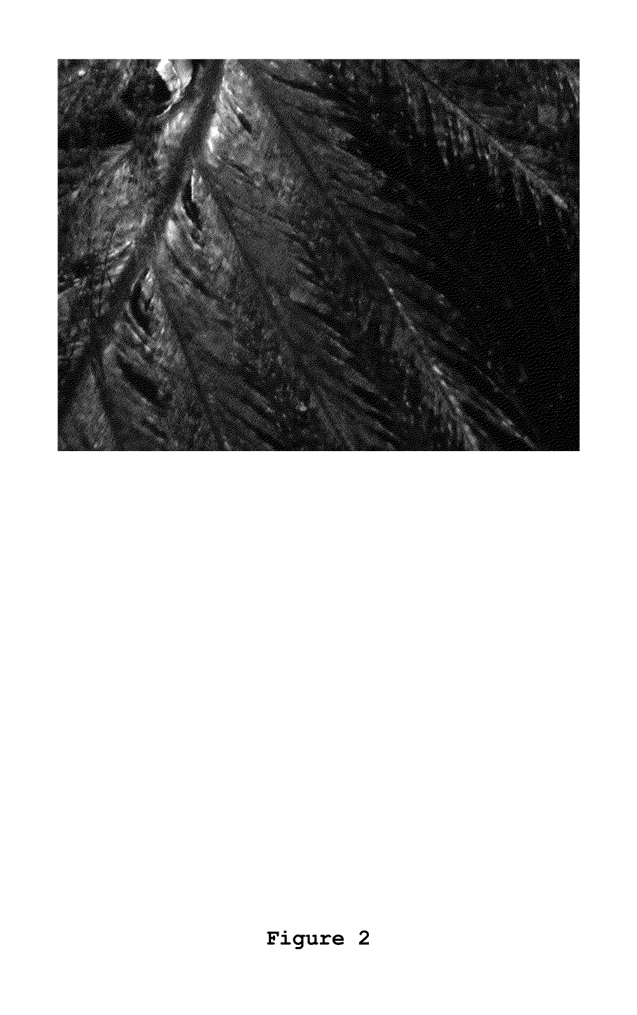

FIG. 2 is an image of a Female Tree Swallow right side scapular feather after exposure to UVA light for one week. As depicted in FIG. 2, there is an increase in the concentration of nanostructures that fluoresce blue. However, there are others, particularly in the shaft of the feather, fluorescing yellow, orange or red indicative of the number and locations of red shifted excitons.

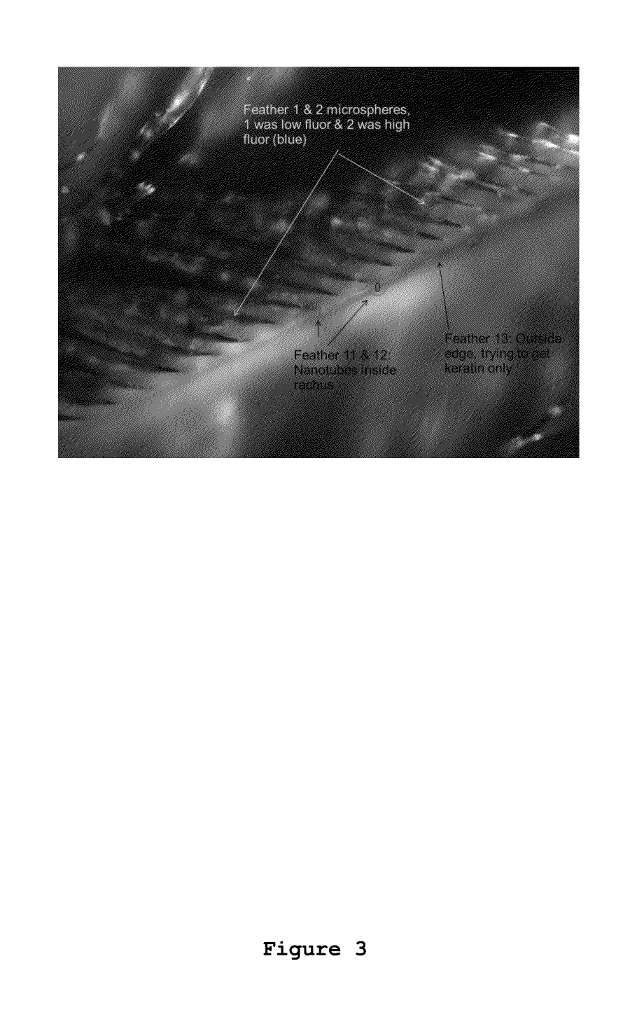

FIG. 3 is an image of a Female Tree Swallow feather after UVA irradiation for one week showing micro-spheres (quantum dots) and nano scale light energy conducting tubes (nanowires). FIG. 3 illustrates that the spherical structures increase with size as exposure time, photolysis of feather keratin, and concentration of nanostructure peptide residues increase. Eventually, microspheres released from feathers adhere to the feather surface and facilitate direct measurement of chemical structures using Raman microscopy.

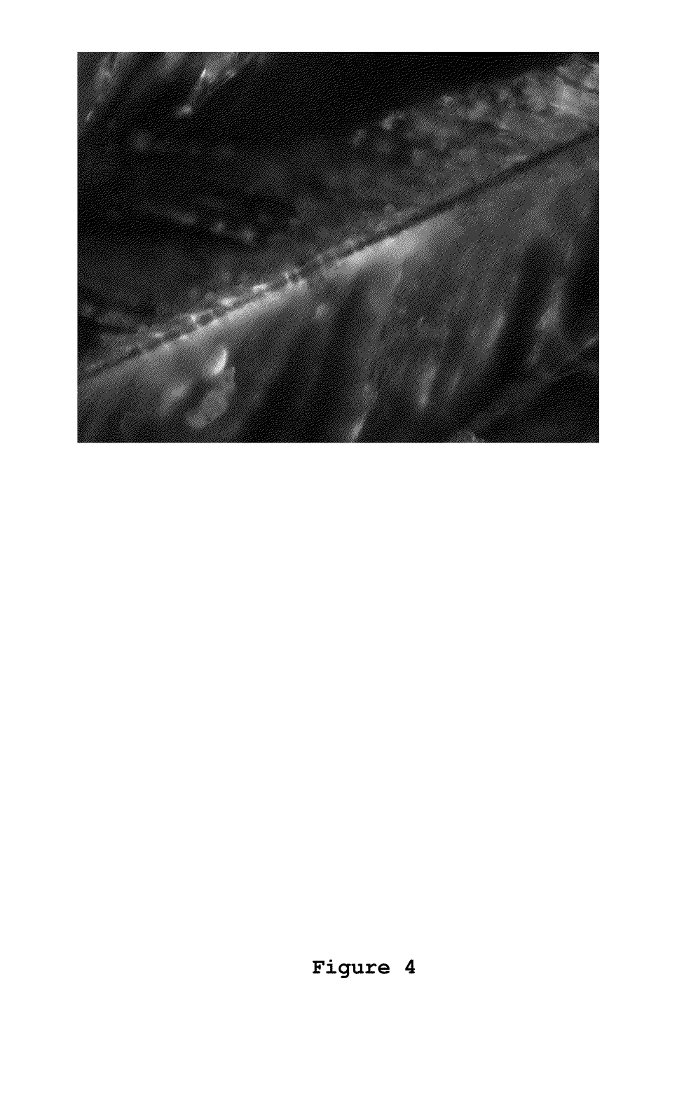

FIG. 4 is an image depicting another focal plane of the same feather and view as illustrated in FIG. 3. The yellow color in the main shaft of the feather indicates the location of nanowires. The wires line the inside of hollow spaces in the feather shaft and in the barbules that connect to the shaft. The nanowires are all interconnected by wires running through holes in the walls of the chambers. The chambers concentrate peptide residues that appear to initially form small quantum dots. As concentrations increase these quantum dots appear to self-assemble into nanowires and, at the highest concentrations, the nanowires self-assemble into microspheres (or large quantum dots shown in blue). If the feather was attached to a living bird, it would not fluoresce as strongly. The red-shifted energy states of the nanostructures in the shaft appear to be saturated and, without a drain on the system (living bird), the nanostructures cannot return to their resting state.

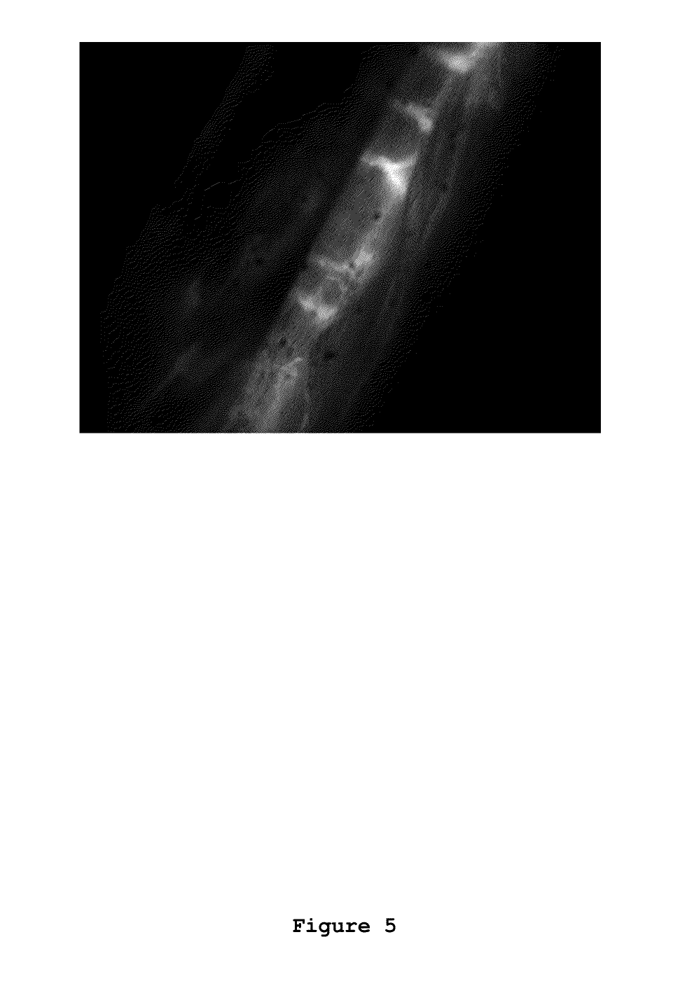

FIG. 5 is an image showing a higher magnification of the feather illustrated in FIGS. 3 and 4. FIG. 5 depicts the network of nanowires in the feather shaft. The nanowires connect to one another across adjacent hollow spaces. The blue fluorescence to the left of the image is of nanowires inside the feather barbule. These nanowires connect to several larger nanowires running along the edges of the barbule (yellow surrounding blue). Such nanowires connect to a bundle of highly concentrated nanowires at the base of the barbule (not shown) where the barbule junctions with the shaft. These bundles connect to nanowires in the shaft, which further connect to larger wires in the main shaft of the feather.

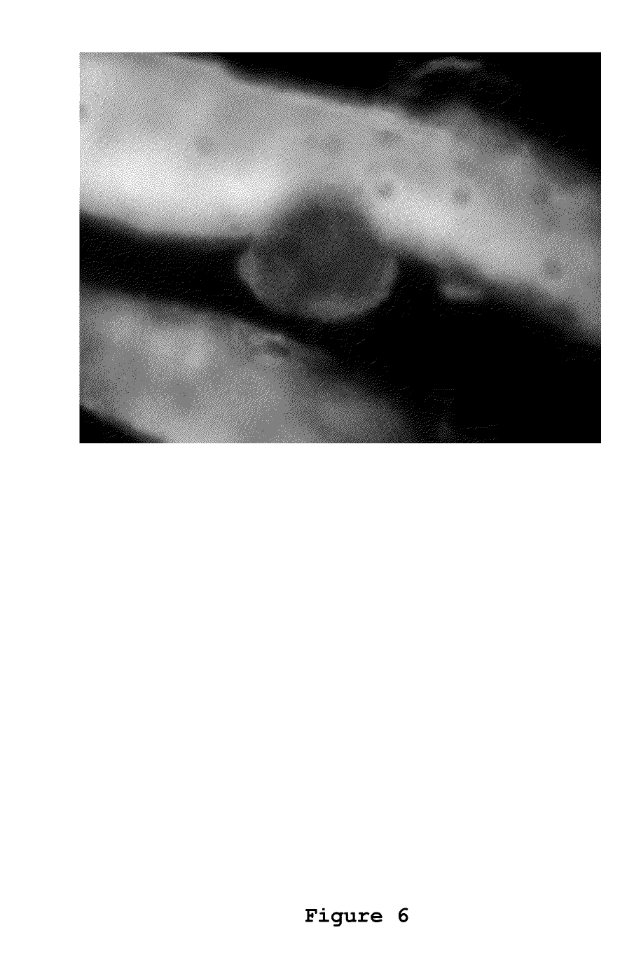

FIG. 6 is an image depicting a microspherical structure (“quantum dot”) in the barbule of the feather depicted in previous figures. The yellow areas are high concentrations of nanowires that have become red-shifted as these energy states have become saturated and cannot return to a resting state. Upon closer examination, blue fluorescent nanowires can be viewed extending from the bottom of the quantum dot and connecting to the bed of nanowires in the barbule (yellow).

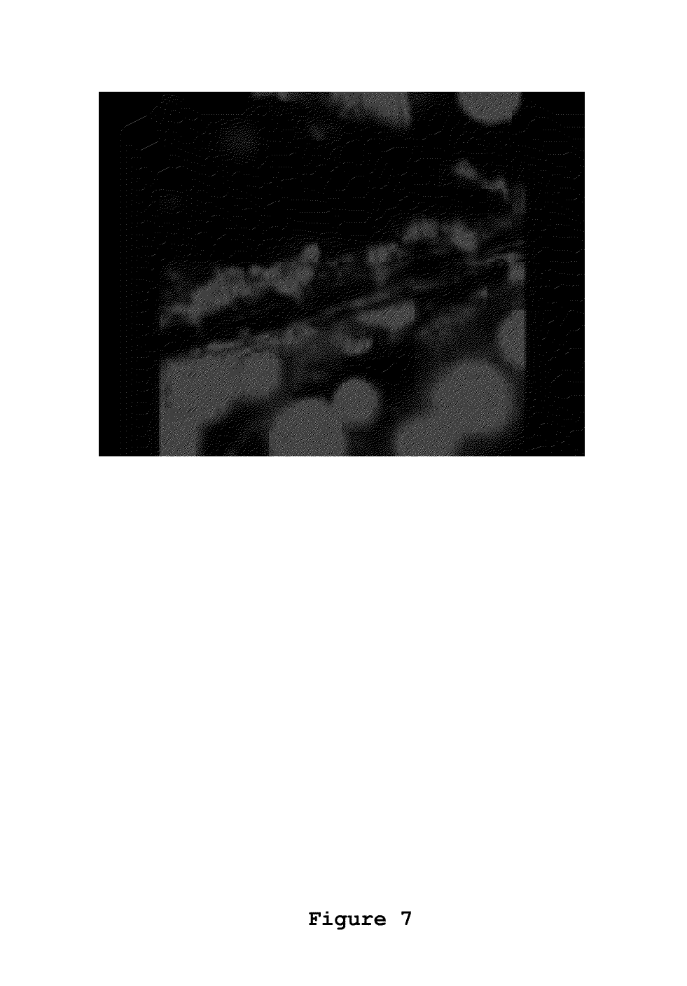

FIG. 7 is a confocal microscopy image with a DAPI filter of the same feather as depicted in previous images, post UVA irradiation for one week. Nanowires can be viewed running from the bottom left up toward the upper right of FIG. 7. These nanowires are in the shaft of the feather barb. The large blue spheres depicted in FIG. 7 are quantum dots in the barbules of the feather (where sunlight harvesting takes place). Along the outer edge of the shaft, concentrations of smaller nanostructures can be seen. Here, nanowires concentrate as they leave the feather barbules, converge, and then reconnect with junctions in the shaft. Current is carried down one side of the shaft to electromagnetic protein receptors in the skin and travel back up the shaft, returning the system to its resting state.

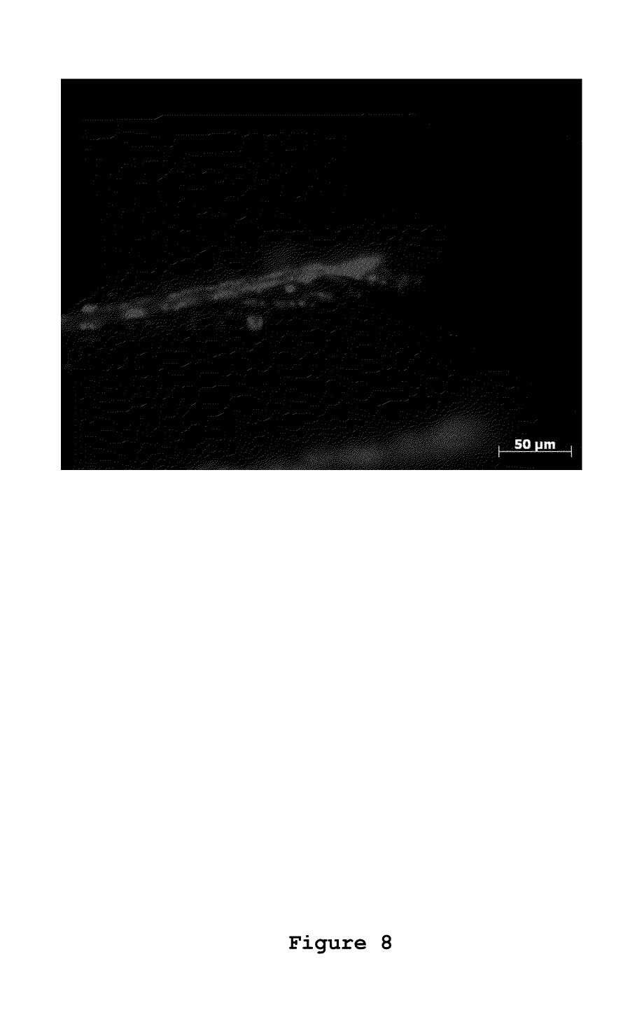

FIG. 8 is an image of a feather barbule shaft with a DAPI filter and UV laser illumination. The two parallel blue lines are nanowires in the shaft. If all wavelengths were visible in this image, one of these wires would be yellow or red fluorescent and the other would be blue. These represent the “source” and “drain” reservoirs that feed electrons to the conducting nano-substrates.

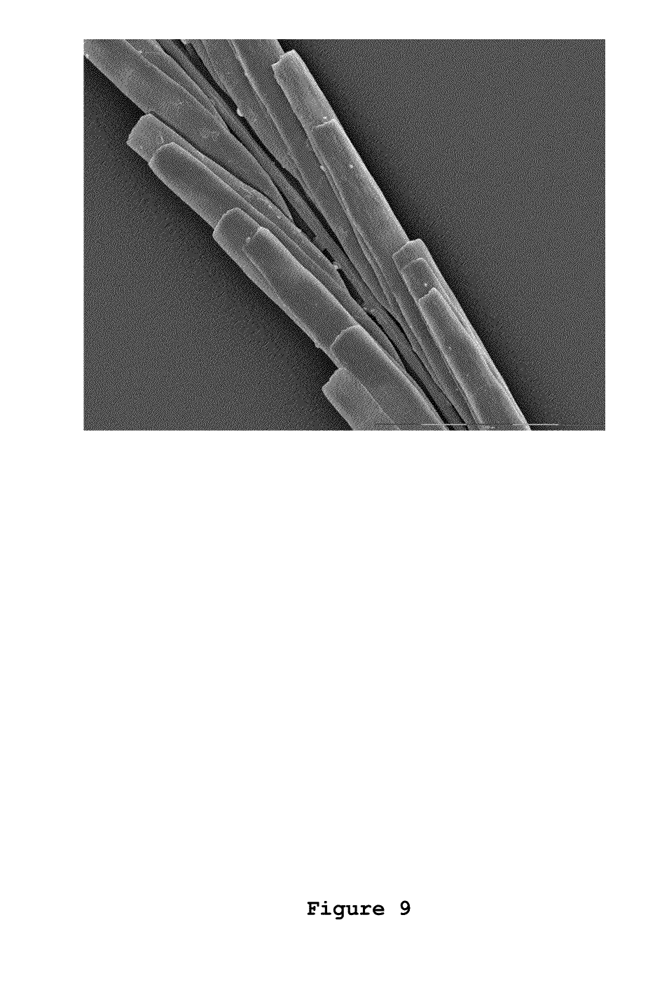

FIG. 9 is a scanning electron microscopy image of a Tree Swallow feather barb. The barb consists of a central shaft and a number of barbules (flattened structures) that connect to the central shaft. This is the upper surface of the feather, or light harvesting surface. The barbules are flattened to increase surface area for light harvesting.

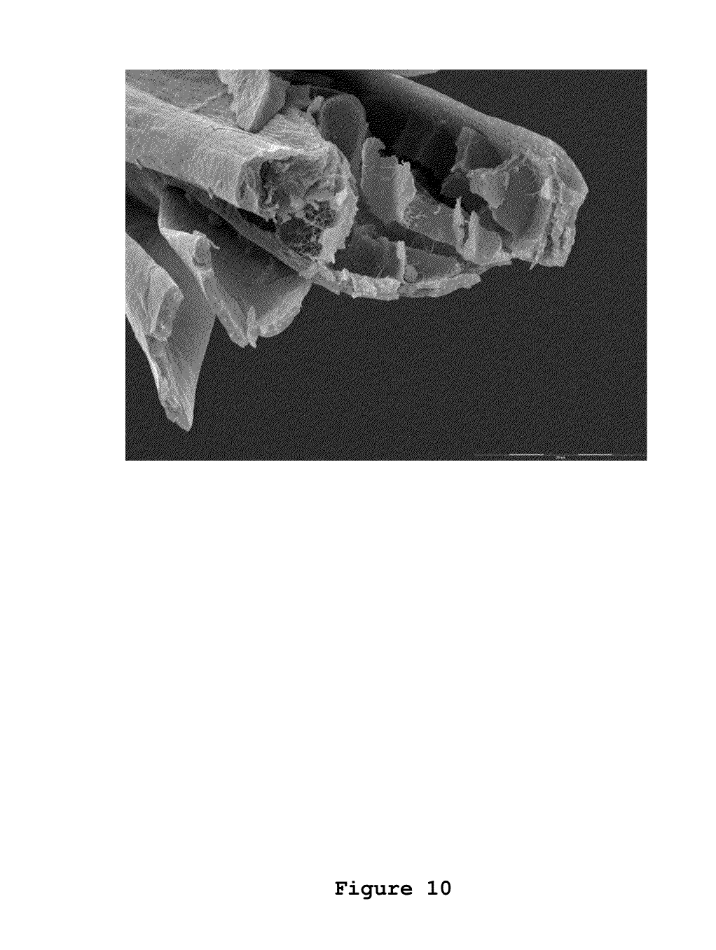

FIG. 10 is a scanning electron microscopy image of a female Tree Swallow feather barb that has been freeze-fractured. Most of the image is of the shaft portion. However, to the left side of the image the base of barbules can be viewed where they attach to the shaft. The ends of the barbules have broken off and are not visible. The hollow areas inside the shaft can be seen as well as the remnants of the walls that divide them. Lining the shaft is a mesh of nanowires. Some individual nanowires can be seen as hair-like filaments inside the hollow spaces of the shaft.

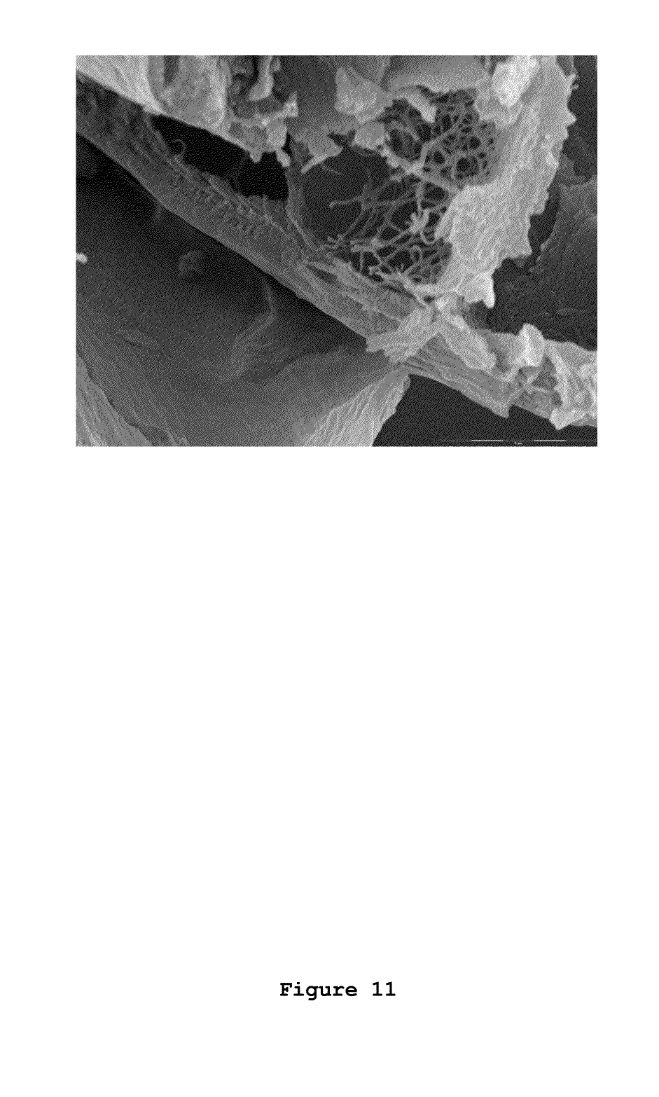

FIG. 11 is an image at higher magnification of the same region of FIG. 10 showing nanowires lining the inside of the feather shaft.

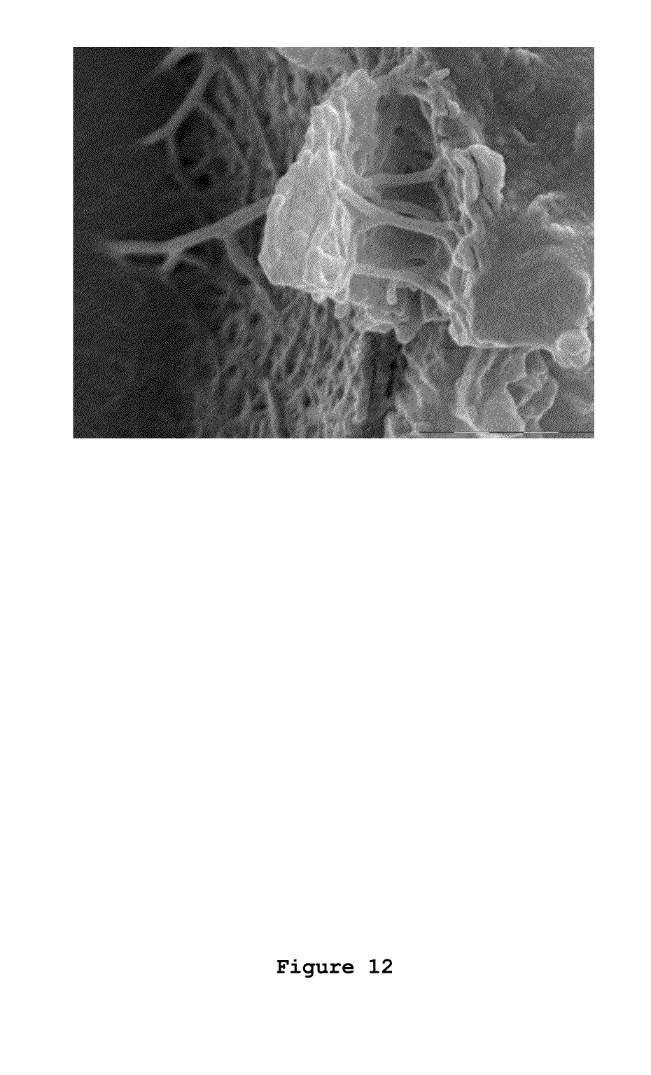

FIG. 12 is an image at higher magnification of nanowires and nanowire mesh that line the inside of a feather shaft (seen on the left of the image). To the right side of the image is the keratinous outer wall of the shaft.

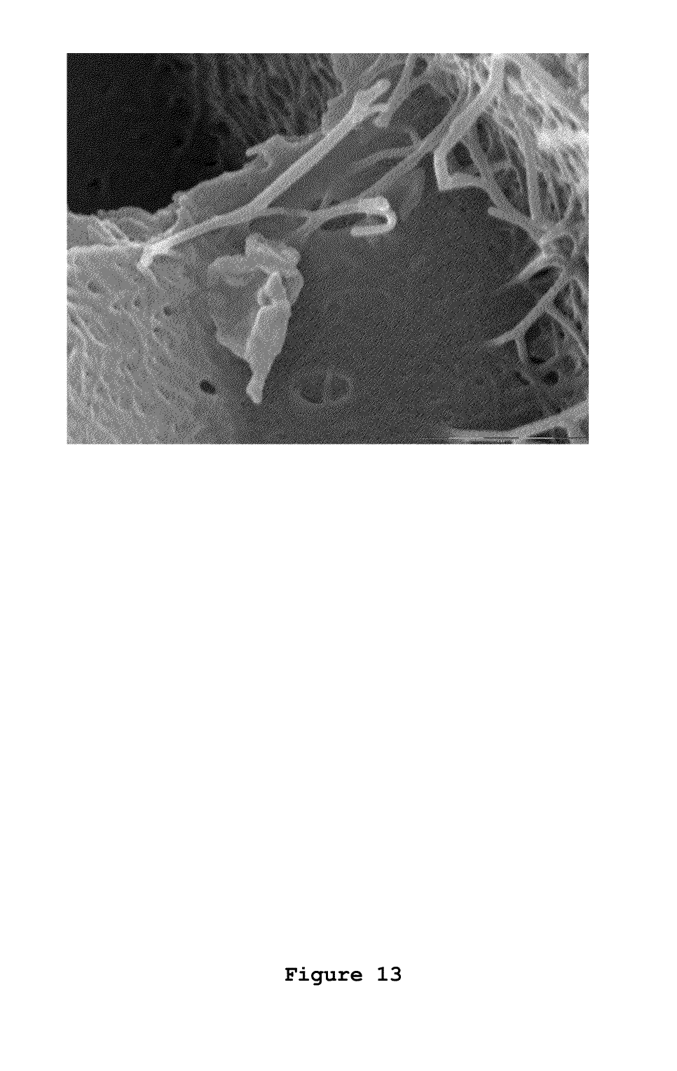

FIG. 13 is another view of nanowires and mesh lining the inside of a feather shaft and barbules.

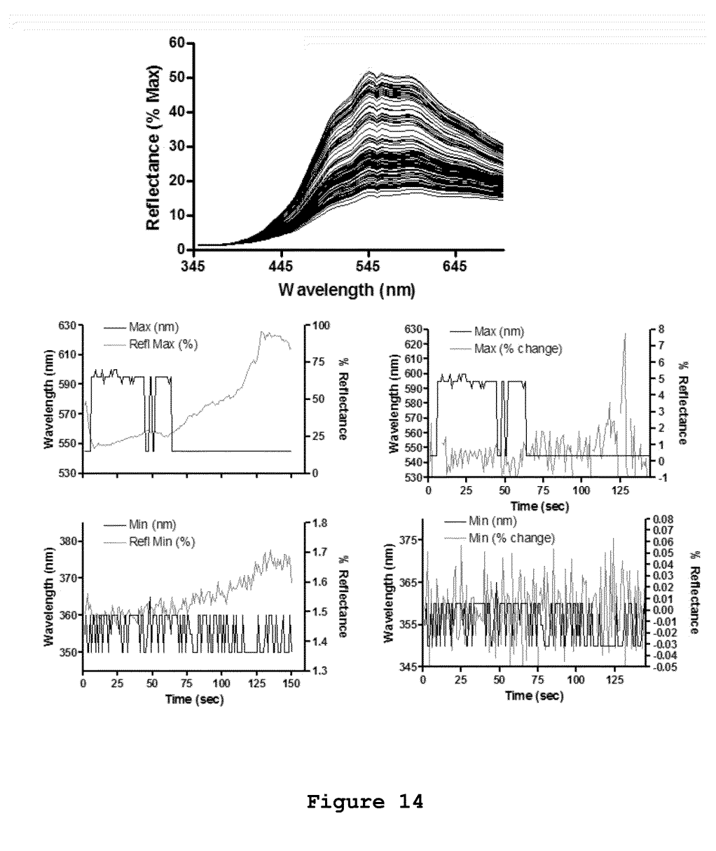

FIG. 14 are graphs depicting Female Tree Swallow crown reflectance and emittance taken at 1 second intervals for 3 minutes illustrating: a) first 70 seconds full spectral data; b) maximum reflectance/emittance and wavelength (nm) over time for 3 minutes; c) percent change from previous measurement in maximum reflectance/emittance over time; d) minimum reflectance/emittance and wavelength (nm) over time for 3 minutes; and e) percent change from previous measurements in minimum reflectance/emittance over time.

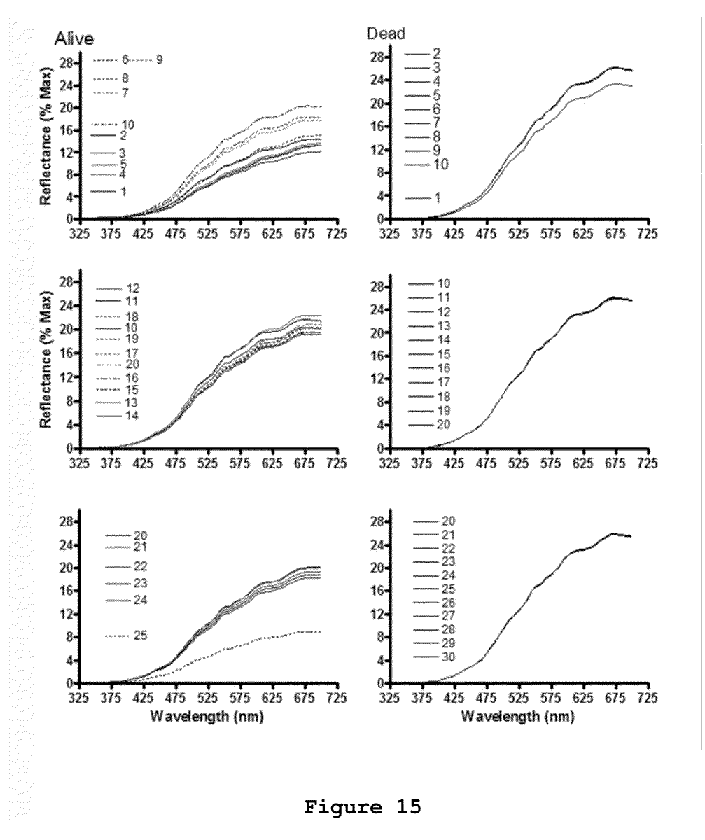

FIG. 15 are graphs depicting current oscillating up and down as indicated by photoluminescence when attached to a living bird (a drain) as compared to a deceased bird. As shown in FIG. 15, the graphs illustrate that light emitted exceeds that of when a bird is alive, indicating that energy is stored when there is no drain on the system.

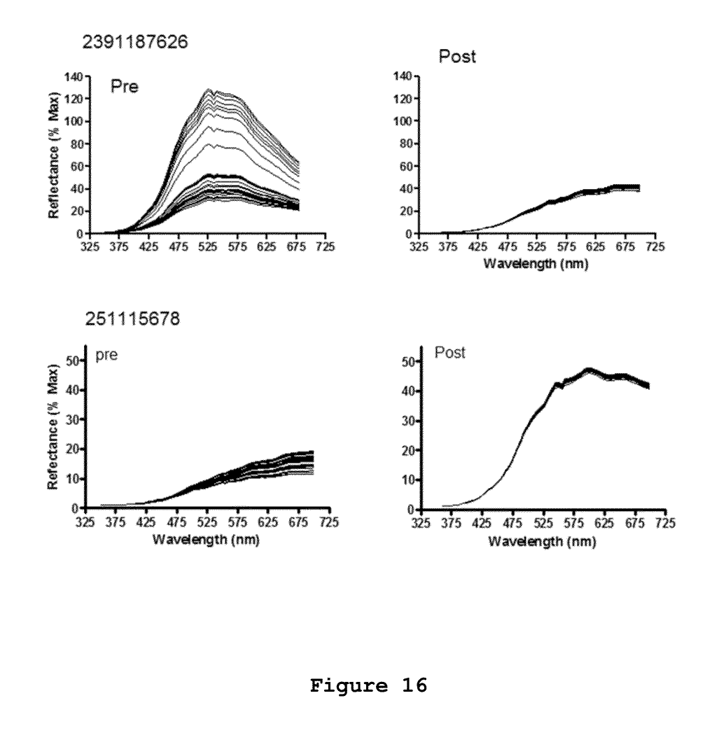

FIG. 16 are examples of pre and post mortem feather photoluminescence indicative of current flow in the system. FIG. 16 illustrates that there is a variation in current when alive and the variation stops post mortem indicating energy is stored.

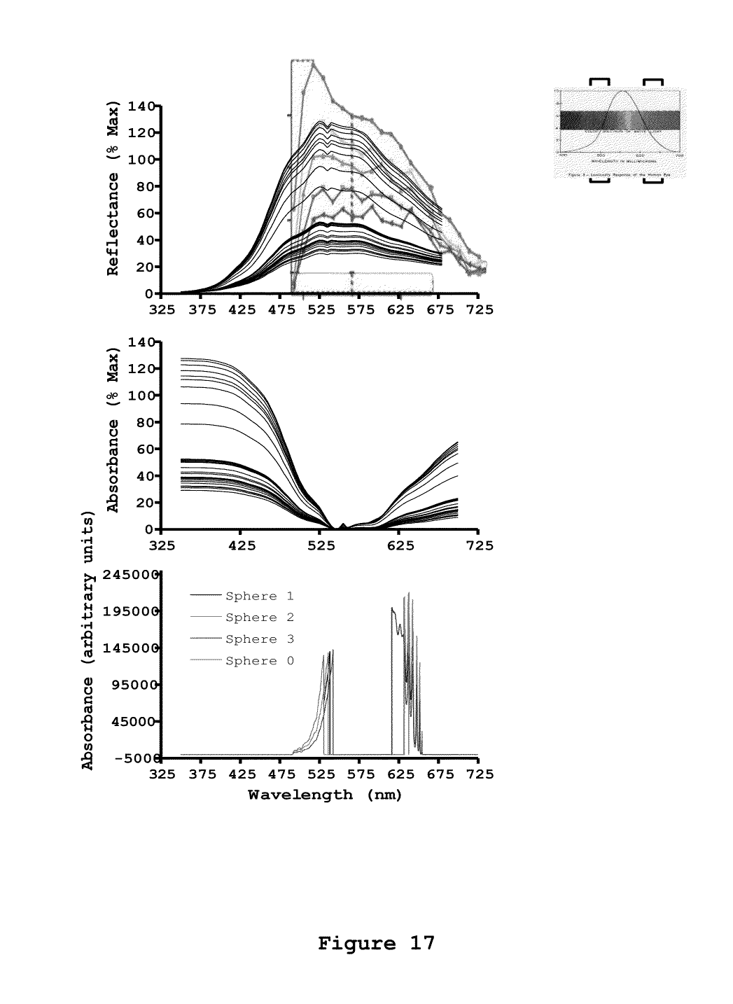

FIG. 17 includes three graphs. The uppermost graph depicts the typical photoluminescence of Tree Swallow feathers when exposed to incident light source continuously for several minutes. The colored lines are the absorbance spectrum for known avian mechanical photoreceptor found in the avian retina that might have been co-opted to receive and regulate signals from light energy propagated by feather-derived nano-structures. The middle graph shows absorbance of Tree Swallow feathers on a live bird collected continuously for several minutes. The bottom graph illustrates absorbance measurements of quantum (or quasi-ballistic) dots excited with a 452 nm laser. The stair-step pattern illustrates the quantum confinement of at least six discrete energy states.

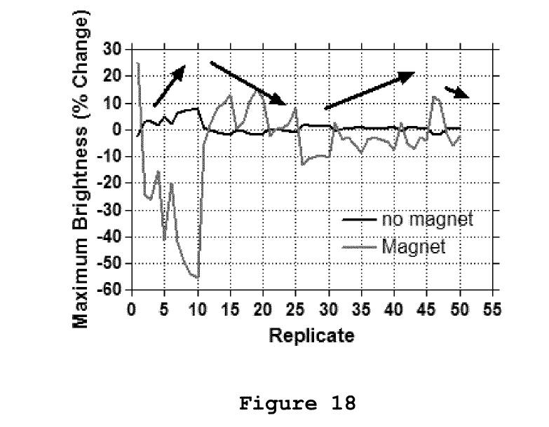

FIG. 18 illustrates how current flow in a live bird is influenced by an electromagnetic field. This is evidence for an electromagnetic switch regulating the amount of solar energy flux in the system. This was performed on a number of birds and pre and post mortem for the same individuals. The magnetic field only has an effect in the live tree swallow, indicating that the system is interacting in a controlled fashion with the living bird through a molecular magnetic switch.

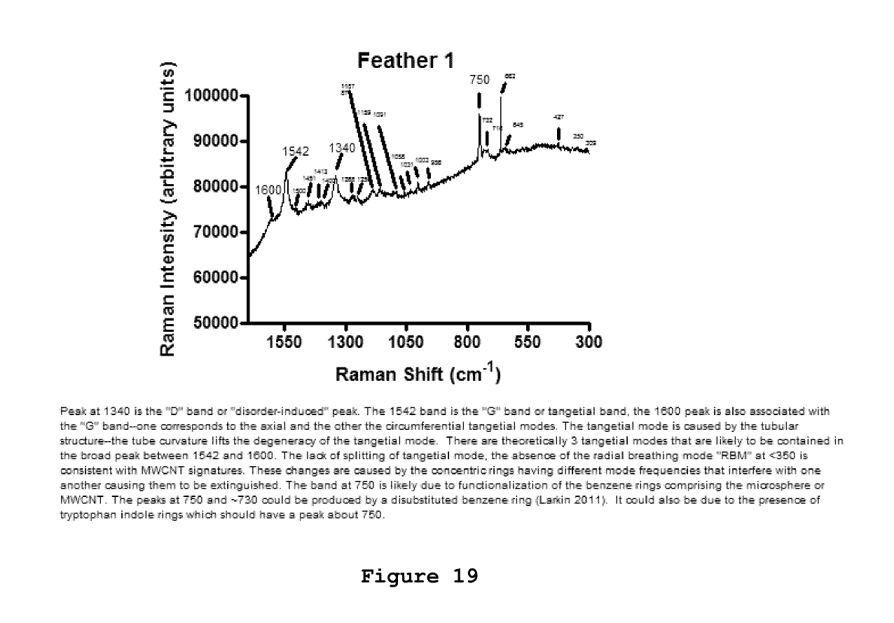

FIG. 19 is raman microscopy of a female Tree Swallow feather with peaks labeled. The profile is indicative of a nano-wire with multiple surface layers that is composed largely of tryptophan residues. Tryptophan is one of the major byproducts of keratin breakdown and fluoresces blue under UVA irradiation.

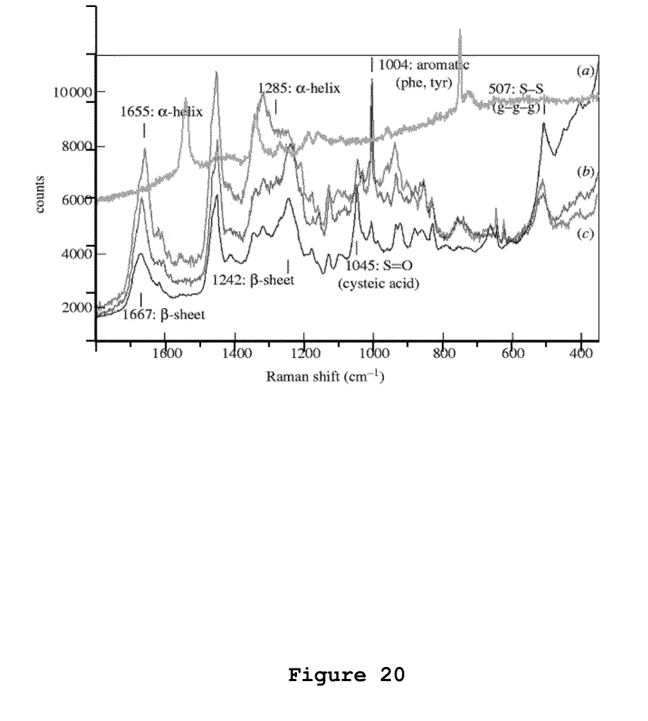

FIG. 20 is a raman microscopy profile comparing nano-wires in a Tree Swallow feather (illustrated in green) to keratin from several taxa. FIG. 20 indicates that the structures are not composed of keratin, but are highly modified and assembled from peptide residues containing mostly tryptophan. There is little evidence depicted in FIG. 20 for phenylalanine or tyrosine, the other major components of keratin oxidation byproducts.

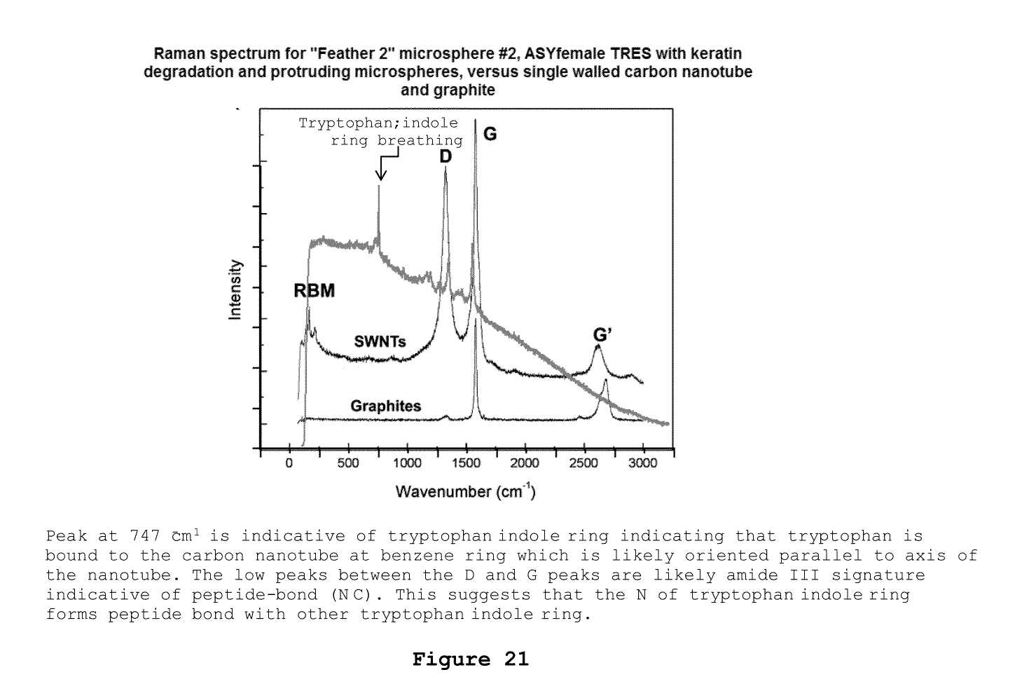

FIG. 21 shows the similarities and differences between nanowire raman scattering profiles and that of carbon nanotubes. It supports the conclusion that they are tubular structures that are multi-walled.

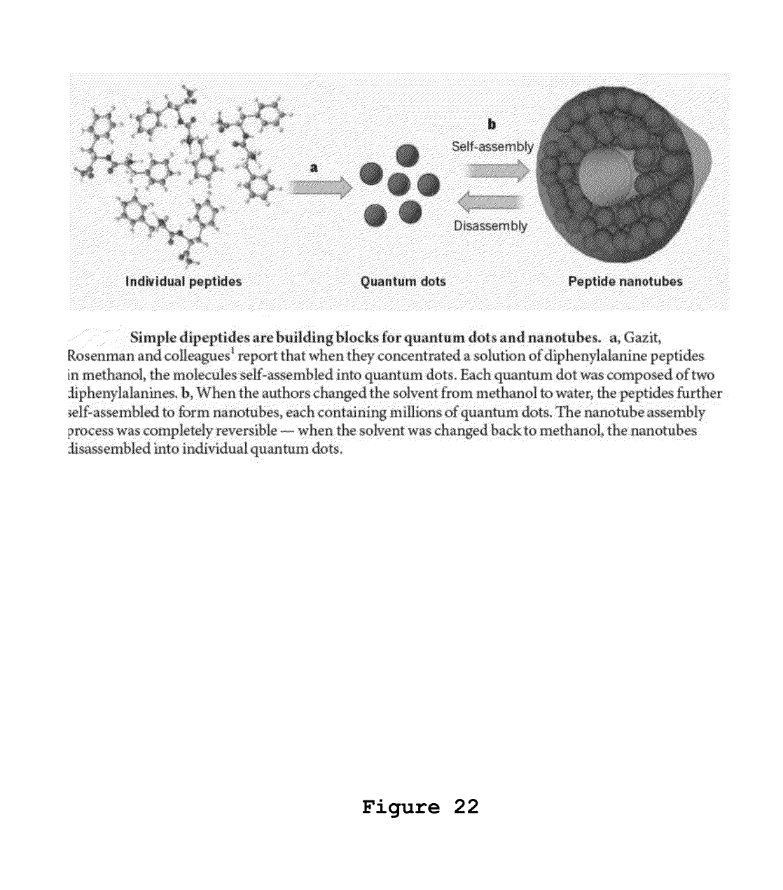

FIG. 22 shows that the alternative to a carbon latticed nanotube configuration is the packing of quantum dots into nanotubes, shown in FIG. 22 for di-phenylalanine. It appears that the nanotubes or nanowires can further assemble into microspheres. Thus, the present invention creates a network of these tubes to harvest and propagate light energy.

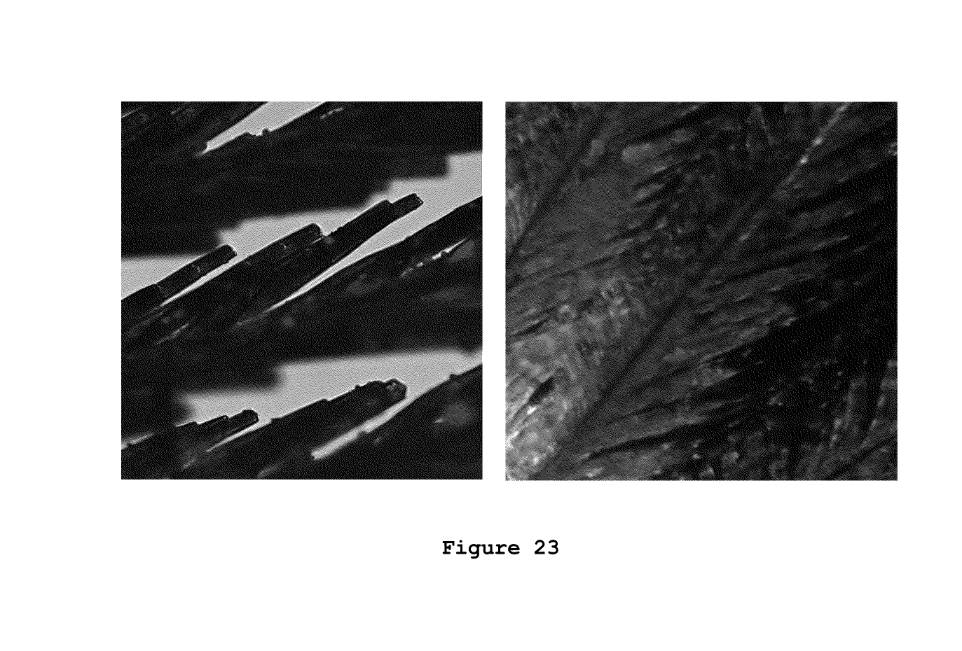

FIG. 23 illustrates Tree Swallow feathers before (left) and after (right) one week of UVA irradiation. After UVA irradiation, nano-structure density and size increased as well as the types of configurations. FIG. 23 demonstrates how the ability of size and density of structures may be used to manipulate light harvesting and propagation characteristics.

The foregoing description has been presented for purposes of illustration and description, and is not intended to be exhausted or to limit the invention to the precise forms disclosed. It is contemplated that other alternative embodiments obvious to those skilled in the art are considered to be included in the invention. The description is merely examples of embodiments. It is understood that many other modifications, substitutions, and additions may be made which are within the intended spirit and scope of the invention. From the foregoing, it can be seen that the present invention accomplishes at least all the stated objections.

What is claimed is:

1. A semi conducting, solar light energy propagating structure comprising: varying diameters that absorbs and propagates solar light over a range of wavelengths; wherein the structure is a reproduced replica of an avian feather.

2. The structure of claim 1, wherein the structure comprises nano wires, nano spheres, microspheres, nano tubes, and quantum dots.

3. The structure of claim 2, wherein the structure is a solar cell.

4. The structure of claim 3, wherein the structure harnesses and propagates light energy via plasmons.

5. An electronic device comprising the structure of claim 2.

6. The electronic device of claim 5, wherein the structure is operatively connected to the electronic device.

7. The electronic device of claim 6, wherein the structure is derived from a keratin substrate.

8. The electronic device of claim 6, wherein the structure is an artificially reproduced replica of an avian feather.

9. The electronic device of claim 5, wherein the electronic device is a quantum computer.

10. A method of energizing a circuit of the electronic device of claim 5, comprising:
extracting a feather from a bird;
removing a keratin substrate from the feather, wherein the keratin substrate comprises the nano wires, nano spheres, microspheres, and nano tubes of the structure;
degrading the feather keratin by ultraviolet irradiation causing the by-products of keratin degradation to increase in concentration;
assembling the by-products into the nano wires, nano spheres, microspheres, and nano tubes of the structure; and
utilizing the nano wires, nano spheres, microspheres, and nano tubes of the structure to propagate energy in a circuit of the electronic device.

11. A method of creating a solar cell, comprising:
organizing from a keratin substrate a series of interconnected hollow chambers that connect to a central shaft similarly comprised of interconnected hollow chambers;
lining the inside of the hollow chambers with nanowires;
passing light through the keratin substrate;
harvesting photocurrent by the nanowires.

12. The method of claim 11, further comprising controlling a flow of electromagnetic energy through the solar cell by a protein molecule switch.

13. The method of claim 12, wherein the solar cell has an energy conversion rate up to 43.5%.

14. The method of claim 12, wherein the solar cell has an energy conversion rate up to 80%.

15. The method of claim 11, wherein the keratin substrate is acquired from wool, feathers, or hair.

16. A method of detecting cancerous tumors in the body, comprising:
forming nano wires, nano spheres, or microspherical structures from a keratin substrate;
applying ultraviolet radiation to the nano wires, nano spheres, and microspherical structures, wherein such structures fluoresce blue light under ultraviolet illumination;
introducing the nano wires, nano spheres, and microspherical structures into the body with suspected cancerous tumors; and
determining the location of cancerous tumors in the body as the structures fluorescing blue light migrate towards areas of the body containing cancerous tumors.

17. The method of claim 16, wherein the nano wires, nano spheres, or microspherical structures formed from the keratin substrate are quantum dots.

18. The method of claim 17, wherein the quantum dots are non-toxic, organic substances.

19. The method of claim 17, wherein the quantum dots encapsulate pharmaceuticals used for cancer treatment, so that when the quantum dots come into contact with a cancerous tumor, the quantum dots provide localized release of the pharmaceutical.

20. The method of claim 19, wherein introducing the quantum dots into the body is via topical solutions administered to the skin and/or hair.
